(19) United States
(12) Patent Application Publication
(10) Pub. No.: US 2014/0099702 A1
(43) Pub. Date: Apr. 10, 2014

(54) DIVIDER FOR USE WITH BIOLISTIC BOMBARDMENT DEVICE

(71) Applicant: E.I. du Pont de Nemours and Company, Wilmington, DE (US)

(72) Inventors: Eric Chuanzhao Li, Wilmington, DE (US); Gregory J. Rairdan, Wilmington, DE (US)

(73) Assignee: E.I. du Pont de Nemours and Company, Wilmington, DE (US)

(21) Appl. No.: 14/105,741

(22) Filed: Dec. 13, 2013

Related U.S. Application Data
(62) Division of application No. 12/980,419, filed on Dec. 29, 2010.
(60) Provisional application No. 61/291,255, filed on Dec. 30, 2009.

(51) Int. Cl. C12N 15/89

(52) U.S. Cl. CPC C12N 15/895
USPC 435/285.3

(57) ABSTRACT

The present invention is designed for use with a biolistic bombardment device having a cold gas shock wave splitter that divides a cold gas shock wave into two or more separate pressure waves that burst into one or more macrocarrier disks so as to create two or more separate microparticle groups. In various embodiments, the present invention provides a divider that is configured to define two or more separate bombardment areas, each configured to contain a respective target and to receive a separate one of the microparticle groups created by a cold gas shock wave splitter. In such a manner, the present invention avoids mixing of microparticles between microparticle groups and allows for independent biolistic bombardment of the targets.

### CROSS-REFERENCE TO RELATED APPLICATIONS

This application is a divisional of U.S. patent application Ser. No. 12/980,419, filed Dec. 29, 2010, which claims priority to U.S. Provisional Application No. 61/291,255, filed Dec. 30, 2009, which applications are hereby incorporated herein by reference in their entirety.

### FIELD

The various embodiments of the present invention generally relate to the genetic engineering of plants. More specifically, embodiments of the present invention relate to a device for improving biolistic plant transformation.

### BACKGROUND

With the rapid advancement of recombinant DNA technology, there is a wide-ranging need for biologists to transfer biologic substances from one cell to another, and to transfer synthetic biological material into living cells to exert their activity therein. Such materials can include biological stains, proteins (antibodies or enzymes), and, most commonly, nucleic acids genetic material (either RNA or DNA). Most of the common techniques are painstakingly slow and use methods which transport materials into, at most, only a few cells at a time. More recently, a biolistic bombardment process has been developed which utilizes a particle gun for microparticle acceleration using gas shock, as described in Sanford, et al., 1987, “Delivery of Substances Into Cells and Tissues Using A Particle Bombardment Process,” Journal of Particle Science and Technology 5:27-37, the disclosure of which is hereby incorporated herein by reference.

The effectiveness of particle transport is measured by the ability of living cells into which the transported particles have been inserted to pick up and express the biological material. This depends upon a wide variety of conditions. The less the expression, the less successful the transport. Correspondingly, the more successful the expression of the living cells (i.e., the extent that they pick up and express the transported biological material), the better the nucleic acid insertion experiments.

In the particle gun technique, biological material (DNA for example) is mixed with a carrier, which may be comprised of a substantially inert metal in the form of small beads that function as microprojectiles that are accelerated using a gas shock wave. Generally, the microprojectiles have a diameter within the range of about 1 micron to about 4 microns and are made from a metal material, such as tungsten, palladium, platinum or gold or an alloy thereof.

Biolistic apparatuses that employ acceleration using gas shock are described, for example, in U.S. Pat. Nos. 5,204,253 and 5,179,022, the disclosures of which are hereby incorporated by reference. A commercially offered version of a biolistic apparatus is the PDS-1000/He™ System available from Bio-Rad Laboratories, Inc. of Hercules, Calif., which uses a high-pressure Helium pulse and a partial vacuum to propel coated microparticles toward target cells in a bombardment chamber. The manufacturer of the PDS-1000/He™ System indicates that the system works as follows: A target containing target cells to be transformed is placed in the bombardment chamber, which is evacuated to subatmospheric pressure. The instrument is then fired allowing Helium to flow into a gas acceleration tube where it is held until the specific pressure of the rupture disk is reached. When the rupture disk bursts, the ensuing Helium shock wave drives a macrocarrier disk, which carries the coated microparticles, a short distance toward a stopping screen. The stopping screen retains the macrocarrier, while the coated microparticles pass through the screen into the bombardment chamber and ultimately penetrate the target cells.

U.S. Pat. No. 5,853,663, the disclosure of which is hereby incorporated by reference, describes an improvement to the above apparatus in the form of a cold gas shock wave splitter whereby a plurality of macrocarriers, and the microcarriers adsorbed onto them, are accelerated towards the target cells. This device effectively spreads out the burst area such that the area is increased compared to the previous apparatus. In particular, the pressure entering the system is split into several separate tubes that supply a plurality of macrocarrier disks held by a macrocarrier plate with fractions of the original pressure burst. This results in a plurality of microprojectile bursts impacting the target cells in an enlarged area.

A commercially offered version of a cold gas shock wave splitter as described above is the Hepta™ Adaptor available from Bio-Rad Laboratories, Inc. of Hercules, Calif. In particular, the Hepta™ Adaptor splits a cold gas shock wave over seven tubes: a central tube and six tubes arranged hexagonally around the central tube. A corresponding macrocarrier plate is included that holds seven macrocarrier disks that are arranged in the same pattern as the seven tubes, with each macrocarrier disk being disposed beneath a respective tube.

With these current systems, however, only one DNA sample can be delivered for each bombardment event. In addition, although cold gas shock wave splitters such as the Hepta™ Adaptor enable more area to be covered than a standard system and maximize the number of cells transformed during one bombardment, if more than one DNA sample is spread over the different macrocarriers, different DNA samples are mixed or overlapped on the targeted tissue. As a result, there is a need for a device configured to prevent the microparticles of a biolistic system from getting mixed or overlapped when using a biolistic bombardment device.

### SUMMARY

The present invention addresses the above needs and achieves other advantages by providing a divider for use with a biolistic bombardment device that includes a cold gas shock wave splitter that divides a cold gas shock wave into two or more separate pressure waves that burst into one or more macrocarrier disks so as to create two or more separate microparticle groups that enter into a bombardment chamber at two or more respective launch areas and that propel toward target cells of two or more individual targets. In general, the divider comprises a base plate configured to support the targets containing the target cells, and at least one dividing wall extending upward from the base plate, wherein a top edge of the at least one dividing wall is positioned between the two or more launch areas, and wherein the base plate and the dividing wall define at least two separate bombardment areas each configured to contain a respective target and to receive one of the separate microparticle groups, thus preventing mixing of the microparticles between the two or more microparticle groups and allowing independent biolistic bombardment of the targets.

In some embodiments, the cold gas shock wave splitter divides the cold gas shock wave into two or more tubes, and the top edge of the dividing wall is positioned below the cold gas shock wave splitter and between the two or more launch areas. In some embodiments, the cold gas shock wave splitter divides the cold gas shock wave into six tubes that are arranged hexagonally, wherein each tube is positioned above one of six macrocarrier disks arranged in the same pattern as the tubes, thus creating six separate microparticle groups that enter into the bombardment chamber at six respective launch areas, and wherein the divider comprises six dividing walls that extend upward from the base plate, and wherein a top edge of each dividing wall is positioned below and between two adjacent launch areas, and wherein the base plate and the dividing walls define six separate bombardment areas each configured to contain a respective target, thus allowing independent biolistic bombardment of the six targets.

In some embodiments, the cold gas shock wave splitter divides the cold gas shock wave into seven tubes comprising a central tube and six perimeter tubes that are arranged hexagonally around the central tube, wherein the six perimeter tubes are positioned above six macrocarrier disks arranged in the same hexagonally arranged pattern, thus creating six separate microparticle groups that enter into the bombardment chamber at six respective launch areas, and wherein the divider comprises six dividing walls that extend upward from the base plate and that are radially disposed about a central divider tube that also extends upward from the base plate, and wherein the central divider tube is substantially aligned and positioned below the central splitter tube, wherein a top edge of each dividing wall is positioned between two adjacent launch areas, and wherein the base plate, central divider tube, and the dividing walls define six separate bombardment areas each configured to contain a respective target, thus allowing independent biolistic bombardment of the six targets.

The present invention also provides a system configured for the independent biolistic bombardment of two or more individual targets containing target cells. In general, the system comprises a biolistic bombardment device including a cold gas shock wave splitter that divides a cold gas shock wave into two or more separate pressure waves that burst into one or more macrocarriers so as to create two or more separate microparticle groups that enter into a bombardment chamber at two or more respective launch areas and that propel toward the target cells, and a divider comprising a base plate configured to support the two or more individual targets containing the target cells and at least one dividing wall extending upward from the base plate, wherein a top edge of the at least one dividing wall is positioned between the two or more launch areas, and wherein the base plate and the dividing wall define at least two separate bombardment areas each configured to contain a respective target and to receive one of the separate microparticle groups, thus preventing mixing of the microparticles between the two or more microparticle groups and allowing independent biolistic bombardment of the targets. In some embodiments, the cold gas shock wave splitter comprises two or more tubes and the top edge of the dividing wall is positioned below the cold gas shock wave splitter and between the two or more launch areas. In some embodiments, the cold gas shock wave splitter comprises six tubes that are arranged hexagonally, wherein each tube is positioned above one of six macrocarriers arranged in the same pattern as the tubes, thus creating six separate microparticle groups that enter into the bombardment chamber at six respective launch areas, wherein the divider comprises six dividing walls that extend upward from the base plate, and wherein a top edge of each dividing wall is positioned below and between two adjacent launch areas, and wherein the base plate and the dividing walls define six separate bombardment areas each configured to contain a respective target, thus allowing independent biolistic bombardment of the six targets.

In some embodiments, the cold gas shock wave splitter comprises seven tubes including a central tube and six perimeter tubes that are arranged hexagonally around the central tube, wherein the six perimeter tubes are positioned above six macrocarrier disks arranged in the same hexagonally arranged pattern, thus creating six separate microparticle groups that enter into the bombardment chamber at six respective launch areas, and wherein the divider comprises six dividing walls that extend upward from the base plate and that are radially disposed about a central divider tube that also extends upward from the base plate, and wherein the central divider tube is positioned substantially aligned and positioned below the central splitter tube, wherein a top edge of each dividing wall is positioned between two adjacent launch areas, and wherein the base plate, central divider tube, and the dividing walls define six separate bombardment areas each configured to contain a respective target, thus allowing independent biolistic bombardment of the six targets.

### BRIEF DESCRIPTION OF THE DRAWINGS

Having thus described the invention in general terms, reference will now be made to the accompanying drawings, which are not necessarily drawn to scale, and wherein:

FIG. 1 shows a front view of a biolistic bombardment device for use with an exemplary embodiment of the present invention;

FIG. 2 shows a cold gas shock wave splitter and a macrocarrier holder of a biolistic bombardment device for use with an exemplary embodiment of the present invention;

FIG. 3 shows a front schematic view of various portions of a biolistic bombardment device for use with an exemplary embodiment of the present invention;

FIG. 4 shows a perspective view of a divider for use with a biolistic bombardment device in accordance with an exemplary embodiment of the present invention;

FIG. 5 shows a top view of a divider containing targets for use with a biolistic bombardment device in accordance with an exemplary embodiment of the present invention;

FIG. 6 shows a front view of a portion of a biolistic bombardment device and a divider in accordance with an exemplary embodiment of the present invention; and

FIG. 7 shows a front schematic view of a portion of a divider and various portions of a biolistic bombardment device in accordance with an exemplary embodiment of the present invention.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

The present invention is designed for use with a biolistic bombardment device having a cold gas shock wave splitter that divides a cold gas shock wave into two or more separate pressure waves that burst into one or more macrocarrier disks so as to create two or more separate microparticle groups. In various embodiments, the present invention provides a divider that is configured to define two or more separate bombardment areas, each configured to contain a respective target and to receive a separate one of the microparticle groups created by a cold gas shock wave splitter. In such a manner, the present invention avoids mixing of microparticles between microparticle groups and allows for independent biolistic bombardment of the targets.

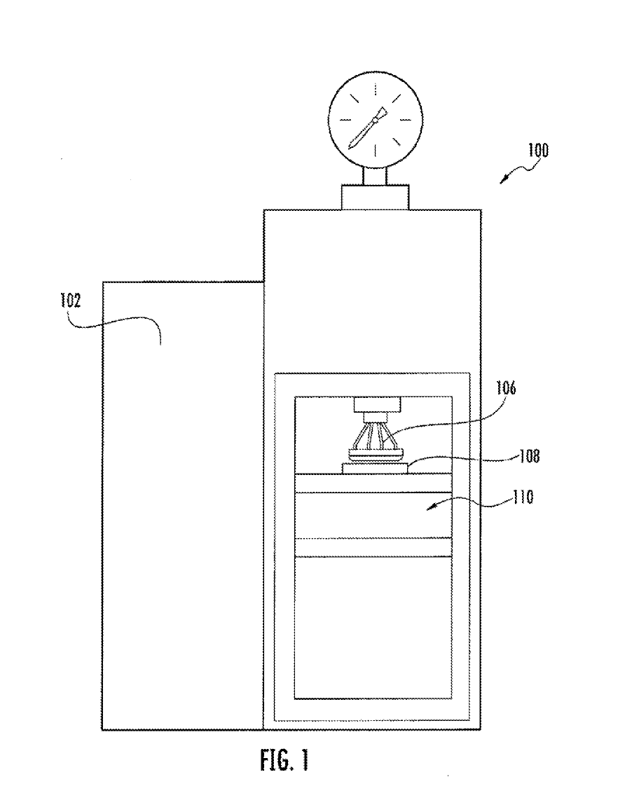

FIG. 1 shows a front view of a biolistic bombardment device 100 for use with an exemplary embodiment of the present invention. Although in various embodiments the present invention may be configured for use with a variety of biolistic bombardment devices, the exemplary embodiment of the present invention is configured for use with a PDS-1000/He™ System available from Bio-Rad Laboratories, Inc. of Hercules, Calif.

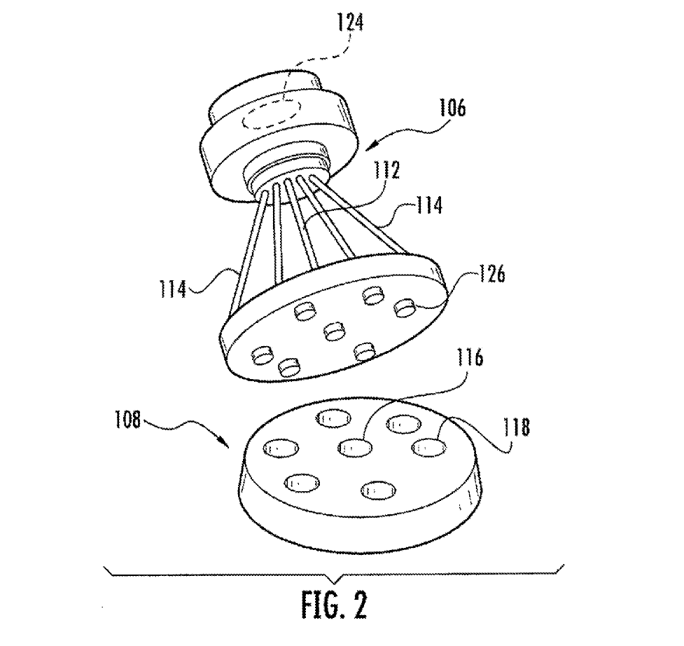

Among the various components of the biolistic bombardment device 100 shown in the figure are a controller 102, a cold gas shock wave splitter 106, a macrocarrier holder 108, and a bombardment chamber 110. In general, the biolistic bombardment device 100 shown in the figure is configured to use a high-pressure Helium pulse and a partial vacuum to propel coated microparticles toward target cells of a target located in the bombardment chamber 110. FIG. 2 shows the cold gas shock wave splitter 106 and the macrocarrier holder 108 of the biolistic bombardment device 100 of FIG. 1. Although in various embodiments the present invention may be configured for use with a variety of cold gas shock wave splitter designs, the exemplary embodiment of the present invention is configured for use with a Hepta™ Adaptor available from Bio-Rad Laboratories, Inc. of Hercules, Calif.

In the depicted embodiment, the cold gas shock wave splitter 106 is configured to receive a cold gas shock wave and to split the cold gas shock wave into seven tubes 112, 114. As shown in FIG. 2, the cold gas shock wave splitter 106 of the depicted embodiment comprises one central tube 112 and six perimeter tubes 114 arranged hexagonally around the central tube 112. Likewise, the macrocarrier holder 108 of the depicted embodiment includes a central macrocarrier holding slot 116 and six perimeter macrocarrier holding slots 118 arranged hexagonally around the central holding slot 116. In various embodiments, the macrocarrier holding slots 116, 118 are configured to hold macrocarrier disks such that when assembled, the macrocarrier disks are located beneath and are substantially aligned with the cold gas shock wave splitter tubes 112, 114. Although other embodiments may utilize the central macrocarrier holding slot 116, in the depicted embodiment the central macrocarrier holding slot 116 is not utilized and thus no macrocarrier disk is loaded in the central macrocarrier holding slot 116. It should also be noted that in various other embodiments of the present invention, the cold gas shock wave splitter and the macrocarrier holder may have a variety of different configurations wherein the cold gas shock wave splitter splits the cold gas shock wave into two or more separate pressure waves. In addition, although the depicted embodiment shows individual macrocarrier disks, in some embodiments there may be a larger common macrocarrier disk that spans across the two or more pressure waves.

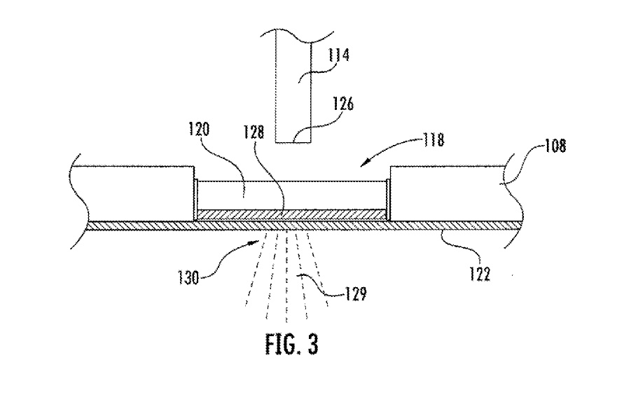

FIG. 3 shows a front schematic view of some portions of the cold gas shock wave splitter 106 and the macrocarrier holder 108 in accordance with an exemplary embodiment of the present invention. In particular, FIG. 3 shows a macrocarrier disk 120 loaded into one of the perimeter holding slots 118 of the macrocarrier holder 108. A stopping screen 122 is also shown located beneath the macrocarrier disk 120. Although the depicted embodiment includes a single stopping screen 122 that extends underneath all of the macrocarrier holding slots 116, 118 of the macrocarrier holder 108, in other embodiments each holding slot may have a separate stopping screen.

Upon firing the biolistic bombardment device 100 of the depicted embodiment, highly pressurized Helium flows into an acceleration chamber of the cold gas shock wave splitter 106, where it is held until the specific pressure of a rupture disk 124 (schematically shown in FIG. 2) is reached. When the rupture disk 124 bursts, the ensuing Helium shock wave enters the tubes 112, 114 of the cold gas shock wave splitter 106 such that the initial wave is split into seven separate pressure waves which travel through the tubes 112, 114 and exit at respective tube ends 126. For the six perimeter macrocarrier slots 118 that have macrocarrier disks 120 loaded therein, the separate shock waves from the cold gas shock wave splitter tubes 114 drive respective macrocarrier disks 120 (which carry coated microparticles 128) toward the stopping screen 122. The stopping screen 122 retains the macrocarrier disks 120, while six separate microparticle groups 129 pass through the screen 122 at six separate launch areas 130 and into the bombardment chamber 110.

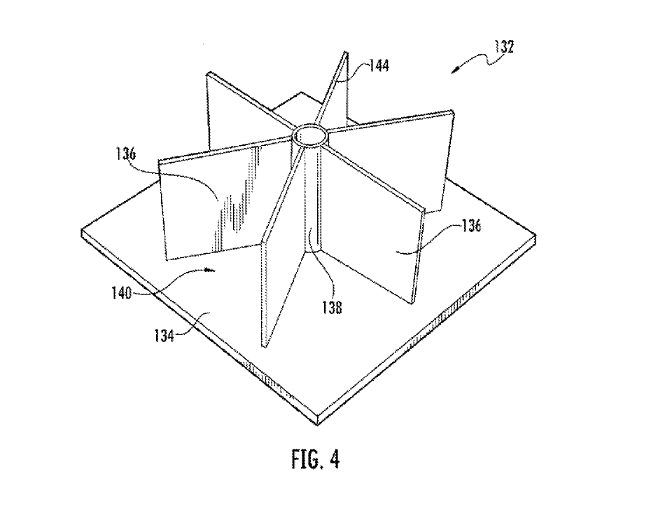

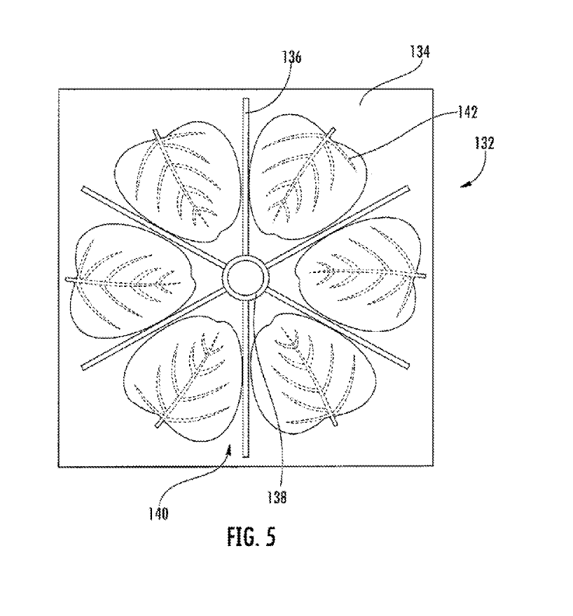

FIG. 4 shows a perspective view of a divider 132 for use with the biolistic bombardment device 100 in accordance with an exemplary embodiment of the present invention. FIG. 5 shows a top view of the divider 132 of FIG. 4, containing six separate targets 142. In the depicted embodiment, the divider 132 comprises a base plate 134 and six dividing walls 136 that extend upward from the base plate 134 and that are radially disposed about a central divider tube 138, which also extends upward from the base plate 134. In such a manner, six separate bombardment areas 140 are created in the divider 132, each of which is configured to contain a separate target 142 and each of which is defined by the base plate 134, the central divider tube 138, and a pair of dividing walls 136. Note that in some embodiments, bombardment areas may be defined by the base plate and at least one dividing wall and thus need not include a central divider tube. In the depicted embodiment, the divider 132 is constructed of a steel material, however in various other embodiments the divider may be constructed of any material configured to create separate bombardment areas, including, but not limited to, other metal materials, plastic materials, composite materials, or combinations thereof. In addition, it should be noted that although in the depicted embodiment the divider 132 has six bombardment areas 140 configured to hold six separate targets 142, in various other embodiments dividers may have two or more bombardment areas configured to contain two or more respective targets.

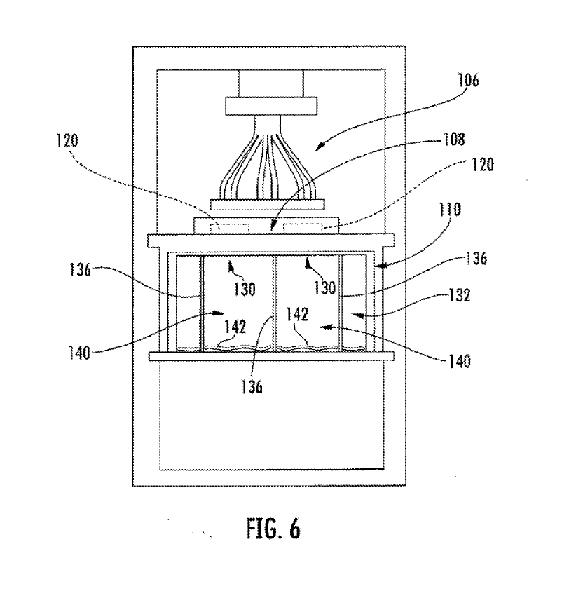

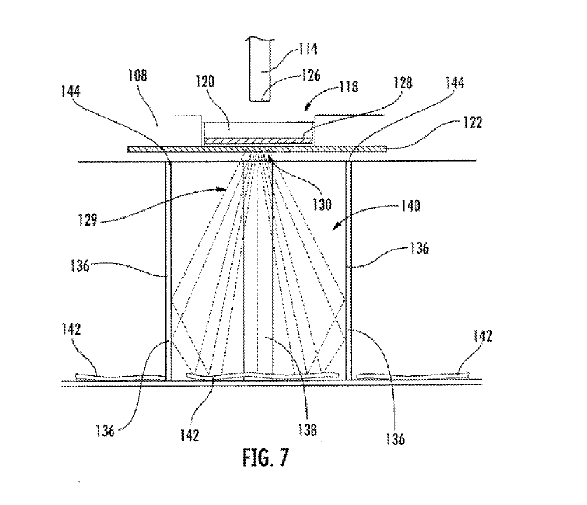

FIG. 6 shows a front view of a portion of the biolistic bombardment device 100 and the divider 132 in accordance with an exemplary embodiment of the present invention. FIG. 7 shows a closer front schematic view of a portion of the divider 132 and various portions of the biolistic bombardment device 100 in accordance with an exemplary embodiment of the present invention. As shown in the figures, the bombardment areas 140 of the divider 132 are generally aligned with the macrocarrier disks 120 and the cold gas shock wave splitter tubes 114. In the depicted embodiment, six targets 142 are placed in the respective bombardment areas 140 of the divider 132 such that each bombardment area 140 receives one target 142. In order to align the divider 132 with the cold gas shock wave splitter 106 and macrocarrier holder 108, the central tube 138 of the divider 132 is aligned below the central macrocarrier holding slot 116 and the central cold gas shock wave splitter tube 112, and a top edge 144 of each dividing wall 136 is positioned between two adjacent launch areas 130. In such a manner, each bombardment area 140 receives a separate microparticle group 129 and mixing of the microparticles between the microparticle groups 129 is prevented, thus allowing independent biolistic bombardment of the targets 142.

It should be noted that in the depicted embodiment the pressure wave exiting from the central cold gas shock wave splitter tube 112 is merely released into the central divider tube 138 and does not affect the surrounding macrocarriers 120 or the targets 142 in the divider 132. In other similar embodiments the central cold gas shock wave splitter tube 112 may be removed or otherwise disabled. However, in embodiments of other configurations, the central area of the divider may be a separate bombardment area and may also hold a target. In such embodiments, the central macrocarrier holding slot 118 may also hold a macrocarrier disk 120.

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1-4. (canceled)

5. A system configured for the independent biolistic bombardment of two or more individual targets containing target cells, the system comprising:
a biolistic bombardment device comprising a cold gas shock wave splitter that divides a cold gas shock wave into two or more separate pressure waves that burst into one or more macrocarriers so as to create two or more separate microparticle groups that enter into a bombardment chamber at two or more respective launch areas and that propel toward the target cells; and
a divider comprising a base plate configured to support the two or more individual targets containing the target cells and at least one dividing wall extending upward from the base plate, wherein a top edge of the at least one dividing wall is positioned between the two or more launch areas, and wherein the base plate and the dividing wall define at least two separate bombardment areas each configured to contain a respective target and to receive one of the separate microparticle groups, thus preventing mixing of the microparticles between the two or more microparticle groups and allowing independent biolistic bombardment of the targets.

6. The system of claim 5, wherein the cold gas shock wave splitter comprises two or more tubes and wherein the top edge of the dividing wall is positioned below the cold gas shock wave splitter and between the two or more launch areas.

7. The system of claim 5, wherein the cold gas shock wave splitter comprises six tubes that are arranged hexagonally, wherein each tube is positioned above one of six macrocarriers arranged in the same pattern as the tubes, thus creating six separate microparticle groups that enter into the bombardment chamber at six respective launch areas, wherein the divider comprises six dividing walls that extend upward from the base plate, and wherein a top edge of each dividing wall is positioned below and between two adjacent launch areas, and wherein the base plate and the dividing walls define six separate bombardment areas each configured to contain a respective target, thus allowing independent biolistic bombardment of the six targets.

8. The system of claim 5, wherein the cold gas shock wave splitter comprises seven tubes including a central tube and six perimeter tubes that are arranged hexagonally around the central tube, wherein the six perimeter tubes are positioned above six macrocarrier disks arranged in the same hexagonally arranged pattern, thus creating six separate microparticle groups that enter into the bombardment chamber at six respective launch areas, and wherein the divider comprises six dividing walls that extend upward from the base plate and that are radially disposed about a central divider tube that also extends upward from the base plate, and wherein the central divider tube is positioned substantially aligned and below the central splitter tube, wherein a top edge of each dividing wall is positioned between two adjacent launch areas, and wherein the base plate, central divider tube, and the dividing walls define six separate bombardment areas each configured to contain a respective target, thus allowing independent biolistic bombardment of the six targets.
